SEQUENCE LISTING (13 sequences)

SEQ ID NO: 1 - protein, 286 aa, Homo sapiens
MSWLLFLAHRVALAALPCRRGSRGFGMFYAVRRGRKTGVFLTWNECRAQVDRFPAARFKKFATEDEAWAFVRKSASPEVSEGHENQHGQESEAKPGKRLREPLDGDGHESAQPYAKHMKPSVEPAPPVSRDTFSYMGDFVVVYTDGCCSSNGRRKPRAGIGVYWGPGHPLNVGIRLPGRQTNQRAEIHAACKAIEQAKTQNINKLVLYTDSMFTINGITNWVQGWKKNGWKTSAGKEVINKEDFVALERLTQGMDIQWMHVPGHSGFIGNEEADRLAREGAKQSED

SEQ ID NO: 2 - protein, 293 aa, Gallus sp.
MLRWLVALLSHSCFVSKGGGMFYAVRKGRQTGVYRTWAECQQQVNRFPSASFKKFATEKEAWAFVGAGPPDGQQSAPAETHGASAVAQENASHREEPETDVLCCNACKRPYEQSTNEEHTVRRAKHDEEQSTPVVSEAKFSYMGEFAVVYTDGCCSGNGRNRARAGIGVYWGPGHPLNISERLPGRQTNQRAEIHAACKAIEQAKSQNIKKLIIYTDSKFTINGITSWVENWKTNGWRTSSGGSVINKEDFQKLDSLSKGIEIQWMHIPGHAGFQGNEEADRLAREGASKQKL

SEQ ID NO: 3 - protein, 348 aa, Saccharomyces cerevisiae
MARQGNFYAVRKGRETGIYNTWNECKNQVDGYGGAIYKKFNSYEQAKSFLGQPNTTSNYGSSTHAGGQVSKPHTTQKRVHRRNRPLHYSSLTSSSACSSLSSANTNTFYSVKSNVPNIESKIFNNWKDCQAYVKHKRGITFKKFEDQLAAENFISGMSAHDYKLMNISKESFESKYKLSSNTMYNKSMNVYCDGSSFGNGTSSSRAGYGAYFEGAPEENISEPLLSGAQTNNRAEIEAVSEALKKIWEKLTNEKEKVNYQIKTDSEYVTKLLNDRYMTYDNKKLEGLPNSDLIVPLVQRFVKVKKYYELNKECFKNNGKFQIEWVKGHDGDPGNEMADFLAKKGASRR

SEQ ID NO: 4 - protein, 155 aa, Escherichia coli
MLKQVEIFTDGSCLGNPGPGGYGAILRYRGREKTFSAGYTRTTNNRMELMAAIVALEALKEHCEVILSTDSQYVRQGITQWIHNWKKRGWKTADKKPVKNVDLWQRLDAALGQHQIKWEWVKGHAGHPENERCDELARAAAMNPTLEDTGYQVEV

SEQ ID NO: 5 - protein, 216 aa, Mus sp.
GICGLGMFYAVRRGRRPGVFLSWSECKAQVDRFPAARFKKFATEDEAWAFVRSSSSPDGSKGQESAHEQKSQAKTSKRPREPLVVVYTDGCCSSNGRKRARAGIGVYWGPGHPLNVRIRLPGRQTNQRAEIHAACKAVMQAKAQNISKLVLYTDSMFTINGITNWVQGWKKNGWRTSTGKDVINKEDFMELDELTQGMDIQWMHIPGHSGFVGNEE

SEQ ID NO: 6 - protein, 286 aa, Homo sapiens
  misc_feature (95)..(96): Xaa can be any naturally occurring amino acid
MSWLLFLAHRVALAALPCRRGSRGFGMFYAVRRGRKTGVFLTWNECRAQVDRFPAARFKKFATEDEAWAFVRKSASPEVSEGHENQHGQESEAKXXKRLREPLDGDGHESAXPYAKHMKPSVEPAPPVSRDTFSYMGDFVVVYTDGCCSSNGRRXPRAGIGVYWGPGHPLNVGIRLPGRQTNQRAEIHAACKAIEQAKTQNINKLVLYTDSMFTINGITNWVQGWKKNGWKTSAGKEVINKEDFVALERLTQGMDIQWMHVPGHSGFIGNEEADRLAREGAKQSED

SEQ ID NO: 7 - DNA, 26 bp, Artificial Sequence
  Synthetic construct
acgctggccgggagtcgaaatgcttc

SEQ ID NO: 8 - DNA, 28 bp, Artificial Sequence
  Synthetic construct
ctgttcctggcccacagagtcgccttgg

SEQ ID NO: 9 - DNA, 29 bp, Artificial Sequence
  Synthetic construct
ggtctttctgacctggaatgagtgcagag

SEQ ID NO: 10 - DNA, 29 bp, Artificial Sequence
  Synthetic construct
cttgcctggtttcgccctccgattcttgt

SEQ ID NO: 11 - DNA, 29 bp, Artificial Sequence
  Synthetic construct
ttgattttcatgcccttctgaaacttccg

SEQ ID NO: 12 - DNA, 34 bp, Artificial Sequence
  Synthetic construct
cctcatcctctatggcaaacttcttaaatctggc

SEQ ID NO: 13 - DNA, 17 bp, Artificial Sequence
  Synthetic construct
gggcgccgtcggtgtgg

(19) United States
(12) Patent Application Publication
(10) Pub. No.: US 2003/0144496 A1
(43) Pub. Date: Jul. 31, 2003

(54) Human RNase H and compositions and uses thereof

(76) Inventors: Stanley T. Crooke, Carlsbad, CA (US); Walter Lima, San Diego, CA (US); Hongjiang Wu, Carlsbad, CA (US)

Correspondence Address: WOODCOCK WASHBURN LLP, ONE LIBERTY PLACE - 46TH FLOOR, PHILADELPHIA, PA, 19103, US

(21) Appl. No.: 10/358,439

(22) Filed: Feb. 3, 2003

Related U.S. Application Data
(63) Continuation-in-part of application No. 09/861,205, filed on May 18, 2001, now abandoned.
Application No. 09/861,205 is a continuation of application No. 09/684,254, filed on Oct. 6, 2000, now Pat. No. 6,376,661.
Application No. 09/684,254 is a continuation of application No. 09/343,809, filed on Jun. 30, 1999, now abandoned.
Application No. 09/343,809 is a continuation of application No. 09/203,716, filed on Dec. 2, 1998, now Pat. No. 6,001,653.
(60) Provisional application No. 60/067,458, filed on Dec. 4, 1997.

(51) Int. Cl. C12Q 1/68; C07H 21/04; C12N 9/22; C12P 21/02; C12N 5/06

(52) U.S. Cl. 536/23.2; 435/6; 435/69.1; 435/320.1; 435/325; 435/199

(57) ABSTRACT

The present invention provides polynucleotides and polypeptides encoded thereby of human Type 2 RNase H. Methods of using these polynucleotides and polypeptides in enhancing antisense oligonucleotide therapies are also provided.

### FIELD OF THE INVENTION

The present invention relates to a human Type 2 RNase H which has now been cloned, expressed and purified to electrophoretic homogeneity and human RNase H and compositions and uses thereof.

This application is a continuation-in-part of U.S. Ser. No. 09/861,205, filed May 18, 2001, which is a continuation of U.S. Ser. No. 09/684,254, filed on Oct. 6, 2000, which is a continuation of U.S. Ser. No. 09/343,809, filed Jun. 30, 1999, which is a continuation of U.S. Ser. No. 09/203,716, filed Dec. 2, 1998 and issued as U.S. Pat. No. 6,001,653 on Dec. 14, 1999 which claims the benefit of priority of U.S. Provisional Application Ser. No. 60/067,458, filed Dec. 4, 1997.

### BACKGROUND OF THE INVENTION

RNase H hydrolyzes RNA in RNA-DNA hybrids. This enzyme was first identified in calf thymus but has subsequently been described in a variety of organisms (Stein, H. and Hausen, P., Science, 1969, 166, 393-395; Hausen, P. and Stein, H., Eur. J. Biochem., 1970, 14, 278-283). RNase H activity appears to be ubiquitous in eukaryotes and bacteria (Itaya, M. and Kondo K. Nucleic Acids Res., 1991, 19, 4443-4449; Itaya et al., Mol. Gen. Genet., 1991, 227, 438-445; Kanaya, S., and Itaya, M., J. Biol. Chem., 1992, 267, 10184-10192; Busen, W., J. Biol. Chem., 1980, 255, 9434-9443; Rong, Y. W. and Carl, P. L., Biochemistry, 1990, 29, 383-389; Eder et al., Biochimie, 1993, 75, 123-126). Although RNases H constitute a family of proteins of varying molecular weight, nucleolytic activity and substrate requirements appear to be similar for the various isotypes. For example, all RNases H studied to date function as endonucleases, exhibiting limited sequence specificity and requiring divalent cations (e.g., Mg2+, Mn2+) to produce cleavage products with 5′ phosphate and 3′ hydroxyl termini (Crouch, R. J., and Dirksen, M. L., Nuclease, Linn, S, M., & Roberts, R. J., Eds., Cold Spring Harbor Laboratory Press, Plainview, N.Y., 1982, 211-241).

In addition to playing a natural role in DNA replication, RNase H has also been shown to be capable of cleaving the RNA component of certain oligonucleotide-RNA duplexes. While many mechanisms have been proposed for oligonucleotide mediated destabilization of target RNAs, the primary mechanism by which antisense oligonucleotides are believed to cause a reduction in target RNA levels is through this RNase H action (Monia et al., J. Biol. Chem., 1993, 268, 14514-14522). In vitro assays have demonstrated that oligonucleotides that are not substrates for RNase H can inhibit protein translation (Blake et al., Biochemistry, 1985, 24, 6139-4145) and that oligonucleotides inhibit protein translation in rabbit reticulocyte extracts that exhibit low RNase H activity. However, more efficient inhibition was found in systems that supported RNase H activity (Walder, R. Y. and Walder, J. A., Proc. Nat'l Acad. Sci. USA, 1988, 85, 5011-5015; Gagnor et al., Nucleic Acid Res., 1987, 15, 10419-10436; Cazenave et al., Nucleic Acid Res., 1989, 17, 4255-4273; and Dash et al., Proc. Nat'l Acad. Sci. USA, 1987, 84, 7896-7900).

Oligonucleotides commonly described as “antisense oligonucleotides” comprise nucleotide sequences sufficient in identity and number to effect specific hybridization with a particular nucleic acid. This nucleic acid or the protein(s) it encodes is generally referred to as the “target.” Oligonucleotides are generally designed to bind either directly to mRNA transcribed from, or to a selected DNA portion of, a pre-selected gene target, thereby modulating the amount of protein translated from the mRNA, or the amount of MRNA transcribed from the gene, respectively. Antisense oligonucleotides may be used as research tools, diagnostic aids, and therapeutic agents.

“Targeting” an oligonucleotide to the associated nucleic acid, in the context of this invention, also refers to a multistep process which usually begins with the identification of the nucleic acid sequence whose function is to be modulated. This may be, for example, a cellular gene (or mRNA transcribed from the gene) whose expression is associated with a particular disorder or disease state, or a foreign nucleic acid from an infectious agent. The targeting process also includes determination of a site or sites within this gene for the oligonucleotide interaction to occur, such that the desired effect, either detection or modulation of expression of the protein, will result.

RNase HI from E. coli is the best-characterized member of the RNase H family. The 3-dimensional structure of E. coli RNase HI has been determined by x ray crystallography, and the key amino acids involved in binding and catalysis have been identified by site-directed mutagenesis (Nakamura et al., Proc. Natl. Acad. Sci. USA, 1991, 88, 11535-11539; Katayanagi et al., Nature, 1990, 347, 306-309; Yang et al., Science, 1990, 249, 1398-1405; Kanaya et al., J. Biol. Chem., 1991, 266, 11621-11627). The enzyme has two distinct structural domains. The major domain consists of four a helices and one large β sheet composed of three antiparallel β strands. The Mg2+ binding site is located on the β sheet and consists of three amino acids, Asp-10, Glu-48, and Gly-11 (Katayanagi et al., Proteins: Struct., Funct., Genet., 1993, 17, 337-346). The structural motif of the Mg2+ binding site surrounded by P strands is similar to that in DNase I (Suck, D., and Oefler, C., Nature, 1986, 321, 620-625). The minor domain is believed to constitute the predominant binding region of the enzyme and is composed of an a helix terminating with a loop. The loop region is composed of a cluster of positively charged amino acids that are believed to bind electrostatistically to the minor groove of the DNA/RNA heteroduplex substrate. Although the conformation of the RNA/DNA substrate can vary from A-form to B-form depending on the sequence composition, in general RNA/DNA heteroduplexes adopt an A-like geometry (Pardi et al., Biochemistry, 1981, 20, 3986-3996; Hall, K. B., and Mclaughlin, L. W., Biochemistry, 1991, 30, 10606-10613; Lane et al., Eur. J. Biochem., 1993, 215, 297-306). The entire binding interaction appears to comprise a single helical turn of the substrate duplex. Recently the binding characteristics, substrate requirements, cleavage products and effects of various chemical modifications of the substrates on the kinetic characteristics of E. coli RNase HI have been studied in more detail (Crooke, S. T. et al., Biochem. J., 1995, 312, 599-608; Lima, W. F. and Crooke, S. T., Biochemistry, 1997, 36, 390-398; Lima, W. F. et al., J. Biol. Chem., 1997, 272, 18191-18199; Tidd, D. M. and Worenius, H. M., Br. J. Cancer, 1989, 60, 343; Tidd, D. M. et al., Anti-Cancer Drug Des., 1988, 3, 117).

In addition to RNase HI, a second E. coli RNase H, RNase HII, has been cloned and characterized (Itaya, M., Proc. Natl. Acad. Sci. USA, 1990, 87, 8587-8591). It is comprised of 213 amino acids, while RNase HI is 155 amino acids long. E. coil RNase II is only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449; Itaya et al., Mol. Gen. Genet., 1991, 227, 438-445). An enzyme cloned from S. cerevisiae was 30% homologous to E. coli RNase HI (Itaya, M. and Kondo K., Nucleic Acids Res., 1991, 19, 4443-4449; Itaya et al., Mol. Gen. Genet., 1991, 227, 438-445). Thus, to date, no enzyme cloned from a species other than E. coli has displayed substantial homology to E. coli RNase H II.

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes, two classes of RNase H have been defined based on differences in molecular weight, effects of divalent cations, sensitivity to sulfhydryl agents and immunological cross-reactivity (Busen et al., Eur. J. Biochem., 1977, 74, 203-208). RNase H Type 1 enzymes are reported to have molecular weights in the 68-90 kDa range, to be activated by either Mn2+ or Mg2+, and to be insensitive to sulfhydryl agents. In contrast, RNase H Type 2 enzymes have been reported to have molecular weights ranging from 31-45 kDa, to require Mg2+, to be highly sensitive to sulfhydryl agents, and to be inhibited by Mn2+ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108).

An enzyme with Type 2 RNase H characteristics has been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5 9. The enzyme requires Mg2+ and is inhibited by Mn2+ and n-ethyl maleimide. The products of cleavage reactions have 3′ hydroxyl and 5′ phosphate termini.

Despite the substantial information about members of the RNase family and the cloning of a number of viral, prokaryotic and yeast genes with RNase H activity, until now, no mammalian RNase H had been cloned. This has hampered efforts to understand the structure, distribution and function of the enzyme(s).

In the present invention, cDNA of human RNase H with Type 2 characteristics and protein expressed thereby are provided.

### SUMMARY OF THE INVENTION

The present invention provides polypeptides which have been identified as novel human Type 2 RNases H by homology between the amino acid sequence set forth in FIG. 1 and known amino acid sequences of chicken, yeast and E. coli RNase HI as well as an expressed sequence tag (EST) deduced mouse RNase H homolog. In accordance with this aspect of the present invention, as a preferred embodiment, a sample of E. coli DH5α containing a BLUESCRIPT™ plasmid containing a human CDNA nucleic acid molecule encoding a human Type 2 RNase H polypeptide, has been deposited as ATCC Deposit No. ATCC 98536.

The present invention also provides polynucleotides that encode human Type 2 RNase H, vectors comprising nucleic acids encoding human RNase H, host cells containing such vectors, antibodies targeted to human Type 2 RNase H, human Type 2 RNase H—his-tag fusion peptides, and nucleic acid probes capable of hybridizing to a nucleic acid encoding a human RNase H polypeptide. Pharmaceutical compositions which include a human Type 2 RNase H polypeptide or a vector encoding a human Type 2 RNase H polypeptide are also provided. These compositions may additionally contain an antisense oligonucleotide.

The present invention is also directed to methods of enhancing antisense inhibition of expression of a target protein via use of human Type 2 RNase H. Methods of screening for effective antisense oligonucleotides and of producing effective antisense oligonucleotides using human Type 2 RNase H are also provided.

Yet another object of the present invention is to provide methods for identifying agents which modulate activity and/or levels of human Type 2 RNase H. In accordance with this aspect, the polynucleotides and polypeptides of the present invention are useful for research, biological and clinical purposes. For example, the polynucleotides and polypeptides are useful in defining the interaction of human Type 2 RNase H and antisense oligonucleotides and identifying means for enhancing this interaction so that antisense oligonucleotides are more effective at inhibiting their target mRNA.

Yet another object of the present invention is to provide a method of prognosticating efficacy of antisense therapy of a selected disease which comprises measuring the level or activity of human RNase H in a target cell of the antisense therapy. Similarly, oligonucleotides can be screened to identify those oligonucleotides which are effective antisense agents by measuring binding of the oligonucleotide to the human Type 2 RNase H.

### BRIEF DESCRIPTION OF THE DRAWINGS

FIG. 1 provides a human Type 2 RNase H primary sequence (286 amino acids; SEQ ID NO: 1) and sequence comparisons with chicken (293 amino acids; SEQ ID NO: 2), yeast (348 amino acids; SEQ ID NO: 3) and E. coli RNase HI (155 amino acids; SEQ ID NO: 4) as well as an EST deduced mouse RNase H homolog (GenBank accession no. AA389926 and AA518920; SEQ ID NO: 5). Boldface type indicates amino acid residues identical to human. “@” indicates the conserved amino acid residues implicated in E. coli RNase HI Mg2+ binding site and catalytic center (Asp- 10, Gly- 11, Glu-48 and Asp-70). “*” indicates the conserved residues implicated in E. coli RNases HI for substrate binding.

### DETAILED DESCRIPTION OF THE INVENTION

A Type 2 human RNase H has now been cloned and expressed. The enzyme encoded by this CDNA is inactive against single-stranded RNA, single-stranded DNA and double-stranded DNA. However, this enzyme cleaves the RNA in an RNA/DNA duplex and cleaves the RNA in a duplex comprised of RNA and a chimeric oligonucleotide with 2′ methoxy flanks and a 5-deoxynucleotide center gap. The rate of cleavage of the RNA duplexed with this so-called “deoxy gapmer” was significantly slower than observed with the full RNA/DNA duplex. These properties are consistent with those reported for E.coli RNase HI (Crooke et al., Biochem. J., 1995, 312, 599-608; Lima, W. F. and Crooke, S. T., Biochemistry, 1997, 36, 390-398). They are also consistent with the properties of a human Type 2 RNase H protein purified from placenta, as the molecular weight (32 kDa) is similar to that reported by Frank et al. (Nucleic Acids Res., 1994, 22, 5247-5254), and the enzyme is inhibited by Mn2+. Accordingly, we refer to the newly cloned human RNase H as Type 2 RNase H or human RNase HI.

Thus, in accordance with one aspect of the present invention, there are provided isolated polynucleotides which encode human Type 2 RNase H polypeptides. By “polynucleotides” it is meant to include any form of RNA or DNA such as mRNA or cDNA or genomic DNA, respectively, obtained by cloning or produced synthetically by well known chemical techniques. DNA may be double- or single-stranded. Single-stranded DNA may comprise the coding or sense strand or the non-coding or antisense strand.

Methods of isolating a polynucleotide of the present invention via cloning techniques are well known. For example, to obtain the cDNA contained in ATCC Deposit No. 98536, primers based on a search of the XREF database were used. An approximately 1-Kb cDNA corresponding to the carboxy terminal portion of the protein was cloned by 3′ RACE. Seven positive clones were isolated by screening a liver cDNA library with this 1-Kb cDNA. The two longest clones were 1698 and 1168 base pairs (bp). They share the same 5′ untranslated region and protein coding sequence but differ in the length of the 3′ UTR. A single reading frame encoding a 286 amino acid protein (calculated mass: 32029.04 Da) was identified. The proposed initiation codon is in agreement with the mammalian translation initiation consensus sequence described by Kozak, M., (J. Cell Biol., 1989, 108, 229-241), and is preceded by an in-frame stop codon. Efforts to clone cDNAs with longer 5′ UTRs from both human liver and lymphocyte cDNAs by 5′ RACE failed, indicating that the 1698 bp clone was full length.

In a preferred embodiment, the polynucleotide of the present invention comprises the nucleic acid sequence of the cDNA contained within ATCC Deposit No. 98536. The deposit of E. coli DH5α containing a BLUESCRIPT™ plasmid containing a human Type 2 RNase H cDNA was made with the American Type Culture Collection, 12301 Park Lawn Drive, Rockville, Md. 20852, USA, on Sep. 4, 1997 and assigned ATCC Deposit No. 98536. The deposited material is a culture of E. coli DH5α containing a BLUESCRIPT™ plasmid (Stratagene, La Jolla, Calif.) that contains the full-length human Type 2 RNase H cDNA. The deposit has been made under the terms of the Budapest Treaty on the international recognition of the deposit of microorganisms for the purposes of patent procedure. The culture will be released to the public, irrevocably and without restriction to the public upon issuance of this patent. The sequence of the polynucleotide contained in the deposited material and the amino acid sequence of the polypeptide encoded thereby are controlling in the event of any conflict with the sequences provided herein. However, as will be obvious to those of skill in the art upon this disclosure, due to the degeneracy of the genetic code, polynucleotides of the present invention may comprise other nucleic acid sequences encoding the polypeptide and derivatives, variants or active fragments thereof.

Another aspect of the present invention relates to the polypeptides encoded by the polynucleotides of the present invention. A polypeptide of the present invention comprises the deduced amino acid sequence of human Type 2 RNase H provided herein as SEQ ID NO: 6. However, by “polypeptide” it is also meant to include fragments, derivatives and analogs which retain essentially the same biological activity and/or function as human Type 2 RNase H. Alternatively, polypeptides of the present invention may retain their ability to bind to an antisense-RNA duplex even though they do not function as active RNase H enzymes in other capacities. In another embodiment, polypeptides of the present invention may retain nuclease activity but without specificity for the RNA portion of an RNA/DNA duplex. Polypeptides of the present invention include recombinant polypeptides, isolated natural polypeptides and synthetic polypeptides, and fragments thereof which retain one or more of the activities described above.

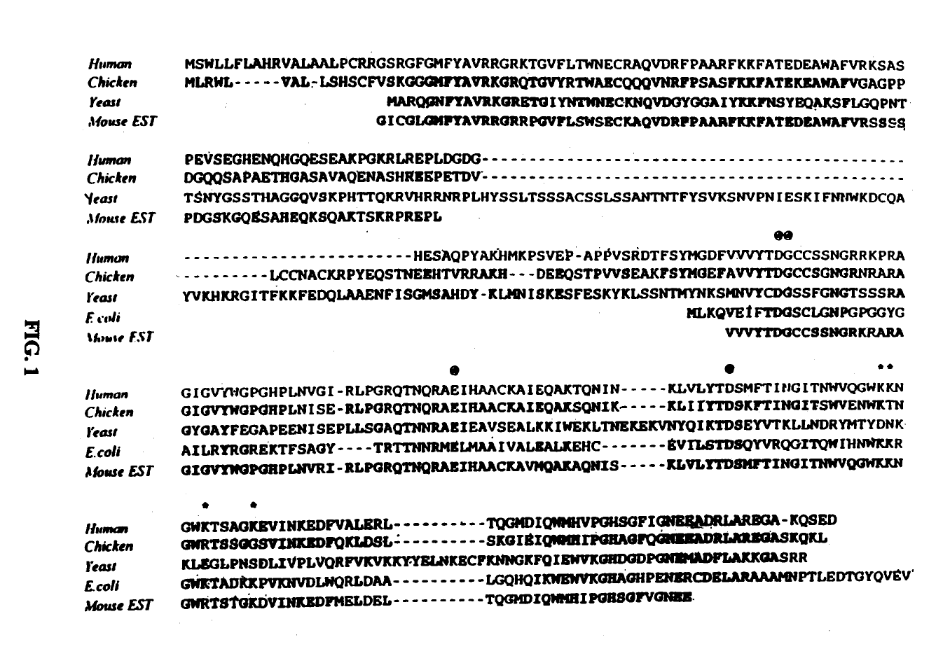

In a preferred embodiment, the polypeptide is prepared recombinantly, most preferably from the culture of E. coli of ATCC Deposit No. 98536. Recombinant human RNase H fused to histidine codons (his-tag; in the present embodiment six histidine codons were used) expressed in E. coli can be conveniently purified to electrophoretic homogeneity by chromatography with Ni-NTA followed by C4 reverse phase HPLC. The polypeptide of SEQ ID NO: 1 is highly homologous to E. coli RNase H, displaying nearly 34% amino acid identity with E. coli RNase HI. FIG. 1 compares a protein sequence deduced from human RNase H cDNA (SEQ ID NO: 1) with those of chicken (SEQ ID NO: 2), yeast (SEQ ID NO: 3) and E. coli RNase HI (GenBank accession no. 1786408; SEQ ID NO: 4), as well as an EST deduced mouse RNase H homolog (GenBank accession no. AA389926 and AA518920; SEQ ID NO: 5). The deduced amino acid sequence of human RNase H (SEQ ID NO: 1) displays strong homology with yeast (21.8% amino acid identity), chicken (59%), E. coli RNase HI (33.6%) and the mouse EST homolog (84.3%). They are all small proteins (<40 KDa) and their estimated pls are all 8.7 and greater. Further, the amino acid residues in E. coli RNase HI thought to be involved in the Mg2+ binding site, catalytic center and substrate binding region are completely conserved in the cloned human RNase H sequence (FIG. 1).

The human Type 2 RNase H is expressed ubiquitously. Northern blot analysis demonstrated that the transcript was abundant in all tissues and cell lines except the MCR-5 line. Northern blot analysis of total RNA from human cell lines and Poly A containing RNA from human tissues using the 1.7 kb full length probe or a 332-nucleotide probe that contained the 5′ UTR and coding region of human RNase H CDNA revealed two strongly positive bands of approximately 1.2 and 5.5 kb in length, and two less intense bands approximately 1.7 and 4.0 kb in length, in most cell lines and tissues. Analysis with the 332-nucleotide probe showed that the 5.5 kb band contained the 5′ UTR and a portion of the coding region, which suggests that this band represents a pre-processed or partially processed transcript, or possibly an alternatively spliced transcript. Intermediate sized bands may represent processing intermediates. The 1.2 kb band represents the full length transcripts. The longer transcripts may be processing intermediates or alternatively spliced transcripts.

RNase H is expressed in most cell lines tested; only MRC5, a breast cancer cell line, displayed very low levels of RNase H. However, a variety of other malignant cell lines including those of bladder (T24), breast (T-47D, HS578T), lung (A549), prostate (LNCap, DU145), and myeloid lineage (HL-60), as well as normal endothelial cells (HUEC), expressed RNase H. Further, all normal human tissues tested expressed RNase H. Again, larger transcripts were present as well as the 1.2 kb transcript that appears to be the mature mRNA for RNase H. Normalization based on G3PDH levels showed that expression was relatively consistent in all of the tissues tested.

The Southern blot analysis of EcoRi digested human and various mammalian vertebrate and yeast genomic DNAs probed with the 1.7 kb probe shows that four EcoRi digestion products of human genomic DNA (2.4, 4.6, 6.0, 8.0 Kb) hybridized with the 1.7 kb probe. The blot re-probed with a 430 nucleotide probe corresponding to the C-terminal portion of the protein showed hybridization of the 430 nucleotide probe to only one 4.6 kb EcoRi digestion product. These data indicate that there is only one gene copy for RNase H and that the size of the gene is more than 10 kb. Both the full length and the shorter probe strongly hybridized to one EcoRI digestion product of yeast genomic DNA (about 5 kb in size), indicating a high degree of conservation. These probes also hybridized to the digestion product from monkey, but none of the other tested mammalian genomic DNAs, including the mouse, which is highly homologous to the human RNase H sequence.

A recombinant human RNase H (his-tag fusion protein) polypeptide of the present invention was expressed in E. coli and purified by Ni-NTA agarose beads followed by C4 reverse phase column chromatography. A 36 kDa protein co-purified with activity measured after renaturation. The presence of the his-tag was confirmed by Western blot analyses with an anti-penta-histidine antibody (Qiagen, Germany).

Renatured recombinant human RNase H displayed RNase H activity. Incubation of 10 ng purified renatured RNase H with RNA/DNA substrate for 2 hours resulted in cleavage of 40% of the substrate. The enzyme also cleaved RNA in an oligonucleotide/RNA duplex in which the oligonucleotide was a 2′-methoxy gapmer with a 5-deoxynucleotide gap, but at a much slower rate than the full RNA/DNA substrate. This is consistent with observations with E. coli RNase HI (Lima, W. F. and Crooke, S. T., Biochemistry, 1997, 36, 390-398). It was inactive against single-stranded RNA or double-stranded RNA substrates and was inhibited by Mn2+. The molecular weight (˜36kDa) and inhibition by Mn2+ indicate that the cloned enzyme is highly homologous to E. coli RNase HI and has properties consistent with those assigned to Type 2 human RNase H.

The sites of cleavage in the RNA in the full RNA/DNA substrate and the gapmer/RNA duplexes (in which the oligonucleotide gapmer had a 5-deoxynucleotide gap) resulting from the recombinant enzyme were determined. In the full RNA/DNA duplex, the principal site of cleavage was near the middle of the substrate, with evidence of less prominent cleavage sites 3′ to the primary cleavage site. The primary cleavage site for the gapmer/RNA duplex was located across the nucleotide adjacent to the junction of the 2′ methoxy wing and oligodeoxynucleotide gap nearest the 3′ end of the RNA. Thus, the enzyme resulted in a major cleavage site in the center of the RNA/DNA substrate and less prominent cleavages to the 3′ side of the major cleavage site. The shift of its major cleavage site to the nucleotide in apposition to the DNA 2′ methoxy junction of the 2′ methoxy wing at the 5′ end of the chimeric oligonucleotide is consistent with the observations for E. coli RNase HI (Crooke et al., Biochem. J., 1995, 312, 599-608; Lima, W. F. and Crooke, S. T., Biochemistry, 1997, 36, 390-398). The fact that the enzyme cleaves at a single site in a 5-deoxy gap duplex indicates that the enzyme has a catalytic region of similar dimensions to that of E. coli RNase HI.

Accordingly, expression of large quantities of a purified human RNase H polypeptide of the present invention is useful in characterizing the activities of a mammalian form of this enzyme. In addition, the polynucleotides and polypeptides of the present invention provide a means for identifying agents which enhance the function of antisense oligonucleotides in human cells and tissues.

For example, a host cell can be genetically engineered to incorporate polynucleotides and express polypeptides of the present invention. Polynucleotides can be introduced into a host cell using any number of well known techniques such as infection, transduction, transfection or transformation. The polynucleotide can be introduced alone or in conjunction with a second polynucleotide encoding a selectable marker. In a preferred embodiment, the host comprises a mammalian cell. Such host cells can then be used not only for production of human Type 2 RNase H, but also to identify agents which increase or decrease levels of expression or activity of human Type 2 RNase H in the cell. In these assays, the host cell would be exposed to an agent suspected of altering levels of expression or activity of human Type 2 RNase in the cells. The level or activity of human Type 2 RNase in the cell would then be determined in the presence and absence of the agent. Assays to determine levels of protein in a cell are well known to those of skill in the art and include, but are not limited to, radioimmunoassay, competitive binding assays, Western blot analysis and enzyme linked immunosorbent assays (ELISAs). Methods of determining increase activity of the enzyme, and in particular increased cleavage of an antisense-mRNA duplex can be performed in accordance with the teachings of Example 5. Agents identified as inducers of the level or activity of this enzyme may be useful in enhancing the efficacy of antisense oligonucleotide therapies.

The present invention also relates to prognostic assays wherein levels of RNase in a cell type can be used in predicting the efficacy of antisense oligonucleotide therapy in specific target cells. High levels of RNase in a selected cell type are expected to correlate with higher efficacy as compared to lower amounts of RNase in a selected cell type which may result in poor cleavage of the MRNA upon binding with the antisense oligonucleotide. For example, the MRC5 breast cancer cell line displayed very low levels of RNase H as compared to other malignant cell types. Accordingly, in this cell type it may be desired to use antisense compounds which do not depend on RNase H activity for their efficacy.

Similarly, oligonucleotides can be screened to identify those which are effective antisense agents by contacting human Type 2 RNase H with an oligonucleotide and measuring binding of the oligonucleotide to the human Type 2 RNase H. Methods of determining binding of two molecules are well known in the art. For example, in one embodiment, the oligonucleotide can be radiolabeled and binding of the oligonucleotide to human Type 2 RNase H can be determined by autoradiography. Alternatively, fusion proteins of human Type 2 RNase H with glutathione-S-transferase or small peptide tags can be prepared and immobilized to a solid phase such as beads. Labeled or unlabeled oligonucleotides to be screened for binding to this enzyme can then be incubated with the solid phase. Oligonucleotides which bind to the enzyme immobilized to the solid phase can then be identified either by detection of bound label or by eluting specifically the bound oligonucleotide from the solid phase. Another method involves screening of oligonucleotide libraries for binding partners. Recombinant tagged or labeled human Type 2 RNase H is used to select oligonucleotides from the library which interact with the enzyme. Sequencing of the oligonucleotides leads to identification of those oligonucleotides which will be more effective as antisense agents.

The following nonlimiting examples are provided to further illustrate the present invention.

### EXAMPLES

### Example 1: Rapid amplification of 5′-cDNA end (5′-RACE) and 3′-cDNA end (3′-RACE)

An internet search of the XREF database in the National Center of Biotechnology Information (NCBI) yielded a 361 bp human EST (GenBank accession #H28861), homologous to yeast RNase H (RNH1) protein sequence tag (EST, GenBank accession #Q04740) and its chicken homologue (accession #D26340). Three sets of oligonucleotide primers encoding the human RNase H EST sequence were synthesized. The sense primers were ACGCTGGCCGGGAGTCGAAATGCTTC (H1: SEQ ID NO: 7), CTGTTCCTGGCCCACAGAGTCGCCTTGG (H3: SEQ ID NO: 8) and GGTCTTTCTGACCTGGAATGAGTGCAGAG (H5: SEQ ID NO: 9). The antisense primers were CTTGCCTGGTTTCGCCCTCCGATTCTTGT (H2: SEQ ID NO: 10), TTGATTTTCATGCCCTTCTGAAACTTCCG (H4; SEQ ID NO: 11) and CCTCATCCTCTATGGCAAACTTCTTAAATCTGGC (H6; SEQ ID NO: 12). The human RNase H 3′ and 5′ cDNAs derived from the EST sequence were amplified by polymerase chain reaction (PCR), using human liver or leukemia (lymphoblastic Molt-4) cell line Marathon ready CDNA as templates, Hi or H3/AP1 as well as H4 or H6/AP2 as primers (Clontech, Palo Alto, Calif.). The fragments were subjected to agarose gel electrophoresis and transferred to nitrocellulose membrane (Bio-Rad, Hercules, Calif.) for confirmation by Southern blot, using 32P labeled H2 and H1 probes (for 3′ and 5′ RACE products, respectively, in accordance with procedures described by Ausubel et al., Current Protocols in Molecular Biology, Wiley and Sons, New York, N.Y., 1988. The confirmed fragments were excised from the agarose gel and purified by gel extraction (Qiagen, Germany), then subcloned into Zero-blunt vector (Invitrogen, Carlsbad, Calif.) and subjected to DNA sequencing.

### Example 2: Screening of the CDNA library, DNA sequencing and sequence analysis

A human liver cDNA lambda phage Uni-ZAP library (Stratagene, La Jolla, Calif.) was screened using the RACE products as specific probes. The positive CDNA clones were excised into the pBluescript phagemid (Stratagene, La Jolla Calif.) from lambda phage and subjected to DNA sequencing with an automatic DNA sequencer (Applied Biosystems, Foster City, Calif.) by Retrogen Inc. (San Diego, Calif.). The overlapping sequences were aligned and combined by the assembling programs of MacDNASIS v3.0 (Hitachi Software Engineering America, South San Francisco, Calif.). Protein structure and subsequence analysis were performed by the program of MacVector 6.0 (Oxford Molecular Group Inc., Campbell, Calif.). A homology search was performed on the NCBI database by internet E-mail.

### Example 3: Northern blot and Southern blot analysis

Total RNA from different human cell lines (ATCC, Rockville, Md.) was prepared and subjected to formaldehyde agarose gel electrophoresis in accordance with procedures described by Ausubel et al., Current Protocols in Molecular Biology, Wiley and Sons, New York, N.Y., 1988, and transferred to nitrocellulose membrane (Bio-Rad, Hercules Calif.). Northern blot hybridization was carried out in QuickHyb buffer (Stratagene, La Jolla, Calif.) with 32P- labeled probe of full length RNase H CDNA clone or primer H1I/H2 PCR-generated 322-base N-terminal RNase H CDNA fragment at 68° C for 2 hours. The membranes were washed twice with 0.1% SSC/0.1% SDS for 30 minutes and subjected to autoradiography. Southern blot analysis was carried out in 1× pre-hybridization/hybridization buffer (BRL, Gaithersburg, Md.) with a 32P-labeled 430 bp C-terminal restriction enzyme PstI/PvuII fragment or 1.7 kb full length cDNA probe at 60° C. for 18 hours. The membranes were washed twice with 0.1% SSC/0.1% SDS at 60° C. for 30 minutes, and subjected to autoradiography.

### Example 4: Expression and purification of the cloned RNase protein

The cDNA fragment coding the full RNase H protein sequence was amplified by PCR using 2 primers, one of which contains restriction enzyme NdeI site adapter and six histidine (his-tag) codons and 22 bp protein N terminal coding sequence. The fragment was cloned into expression vector pETI7b (Novagen, Madison, Wis.) and confirmed by DNA sequencing. The plasmid was transfected into E. coli BL21(DE3) (Novagen, Madison, Wis.). The bacteria were grown in M9ZB medium at 32° C. and harvested when the OD600 of the culture reached 0.8, in accordance with procedures described by Ausubel et al., Current Protocols in Molecular Biology, Wiley and Sons, New York, N.Y., 1988. Cells were lysed in 8M urea solution and recombinant protein was partially purified with Ni-NTA agarose (Qiagen, Germany). Further purification was performed with C4 reverse phase chromatography (Beckrnan, System Gold, Fullerton, Calif.) with 0.1% TFA water and 0.1% TFA acetonitrile gradient of 0% to 80% in 40 minutes as described by Deutscher, M. P., Guide to Protein Purification, Methods in Enzymology 182, Academic Press, New York, N.Y., 1990. The recombinant proteins and control samples were collected, lyophilized and subjected to 12% SDS-PAGE as described by Ausubel et al., Current Protocols in Molecular Biology, Wiley and Sons, New York, N.Y., 1988. The purified protein and control samples were resuspended in 6 M urea solution containing 20 mM Tris HCl, pH 7.4, 400 mM NaCl, 20% glycerol, 0.2 mM PMSF, 5 mM DTT, 10 μg/ml aprotinin and leupeptin, and refolded by dialysis with decreasing urea concentration from 6 M to 0.5 M as well as DTT concentration from 5 mM to 0.5 mM as described by Deutscher, M. P., Guide to Protein Purification, Methods in Enzymology 182, Academic Press, New York, N.Y., 1990. The refolded proteins were concentrated (10 fold) by Centricon (Amicon, Danvers, Mass.) and subjected to RNase H activity assay.

### Example 5: RNase H activity assay

32P-end-labeled 17-mer RNA, GGGCGCCGTCGGTGTGG (SEQ ID NO: 13) described by Lima, W. F. and Crooke, S. T., Biochemistry, 1997, 36, 390-398, was gel-purified as described by Ausubel et al., Current Protocols in Molecular Biology, Wiley and Sons, New York, N.Y., 1988, and annealed with a tenfold excess of its complementary 17-mer oligodeoxynucleotide or a 5-base DNA gapmer, i.e., a 17mer oligonucleotide which has a central portion of 5 deoxynucleotides (the “gap”) flanked on both sides by 6 2′-methoxynucleotides. Annealing was done in 10 mM Tris HCl, pH 8.0, 10 mM MgCl, 50 mM KCl and 0.1 mM DTT to form one of three different substrates: (a) single strand (ss) RNA probe, (b) full RNA/DNA duplex and (c) RNA/DNA gapmer duplex. Each of these substrates was incubated with protein samples at 37° C. for 5 minutes to 2 hours at the same conditions used in the annealing procedure and the reactions were terminated by adding EDTA in accordance with procedures described by Lima, W. F. and Crooke, S. T., Biochemistry, 1997, 36, 390-398. The reaction mixtures were precipitated with TCA centrifugation and the supernatant was measured by liquid scintillation counting (Beckman LS6000IC, Fullerton, Calif.). An aliquot of the reaction mixture was also subjected to denaturing (8 M urea) acrylamide gel electrophoresis in accordance with procedures described by Lima, W. F. and Crooke, S. T., Biochemistry, 1997, 36, 390-398 and Ausubel et al., Current Protocols in Molecular Biology, Wiley and Sons, New York, N.Y., 1988.

What is claimed is:

1. An isolated polynucleotide encoding the human Type 2 RNase H polypeptide (SEQ ID NO: 6):

2. A vector comprising a nucleic acid encoding the human Type 2 RNase H (SEQ ID NO: 6).

3. A host cell comprising the vector of claim 2.

4. A composition comprising a vector comprising a nucleic acid encoding the human Type 2 RNase H polypeptide (SEQ ID NO: 6).

5. The composition of claim 4 further comprising an antisense oligonucleotide.

6. A composition comprising an antisense oligonucleotide and a human Type 2 RNase H polypeptide, wherein the human Type 2 RNase H polypeptide is the human Type 2 RNase H polypeptide (SEQ ID NO: 6).

7. A human Type 2 RNase H--his-tag fusion polypeptide, wherein the human Type 2 RNase H polypeptide is the human Type 2 RNase H polypeptide (SEQ ID NO: 6).

8. A method of identifying an effective antisense oligonucleotide for inhibition of expression of a selected target protein comprising:
(a) contacting the human Type 2 RNase H polypeptide (SEQ ID NO: 6) with an RNA encoding the selected target protein and an oligonucleotide complementary to at least a portion of the RNA under conditions in which an RNA-oligonucleotide duplex is formed;
(b) detecting one or more RNA cleavage products wherein cleavage products of the RNA of the RNA-oligonucleotide duplex are indicative of antisense efficacy.

9. The method of claim 8 further comprising determining the site on the RNA at which cleavage occurs, whereby said site is identified as a Type 2 RNase H-sensitive site.

10. The method of claim 9 further comprising identifying an effective antisense oligonucleotide which hybridizes to said Type 2 RNase H-sensitive site.

11. The method of claim 8 wherein the oligonucleotide is one of a mixture or library of oligonucleotides.

12. A method of identifying agents which increase or decrease activity or levels of the human Type 2 RNase H polypeptide (SEQ ID NO: 6) in a host cell comprising:
(a) contacting a cell in vitro expressing the human Type 2 RNase H polypeptide with an agent suspected of increasing or decreasing activity or levels of the human Type 2 RNase H polypeptide; and
(b) measuring the activity or levels of the human Type 2 RNase H polypeptide in the presence and absence of the agent so that an increase or decrease in the activity or levels of the human Type 2 RNase H polypeptide can be determined.
